(19) United States
(12) Patent Application Publication
(10) Pub. No.: US 2021/0358611 A1
(43) Pub. Date: Nov. 18, 2021

(54) Method for Detecting Epileptic Spike, Method for Training Network Model, and Computer Device

(71) Applicant: Beijing Intelligent Brain Cloud Inc., Beijing (CN)

(72) Inventors: Pan LIAO, Beijing (CN); Li ZHENG, Beijing (CN); Jia-Hong GAO, Beijing (CN)

(21) Appl. No.: 16/997,225

(22) Filed: Aug. 19, 2020

Related U.S. Application Data
(60) Provisional application No. 63/034,494, filed on Jun. 4, 2020.

(30) Foreign Application Priority Data
May 14, 2020 (CN) 202010407992.5

(51) Int. Cl. G16H 40/63; G06N 3/08

(52) U.S. Cl. CPC G16H 40/63; G06N 3/08

(57) ABSTRACT

A method for detecting an epileptic spike includes: obtaining, by a first module of a network model, a local feature of data to be detected, and obtaining, by a second module of the network model, a global feature of the data to be detected; and determining, by a third module of the network model, a detection result of whether there is the epileptic spike in the data to be detected according to the local feature and the global feature. The data to be detected contains a temporal domain and a spatial domain represented by multiple channels, the local feature is a single channel feature, and the global feature is a multichannel feature.

### CROSS-REFERENCE TO RELATED APPLICATIONS

This application claims priority to Provisional Application No. 63/034,494 filed on Jun. 4, 2020, and to Chinese patent application No. 202010407992.5 filed on May 14, 2020. Both applications are incorporated herein by reference in their entireties.

### TECHNICAL FIELD

The present application relates to the field of deep learning technologies, in particular to a method for detecting an epileptic spike, a method for training a network model and a computer device.

### BACKGROUND

As one of the most common neurological brain diseases, epilepsy caused by recurrent seizures affects about 1% of the world's population, and almost 30% to 40% of patients are nonresponsive to medication, resulting in significant negative impacts on their physical, psychological and social health. Surgical intervention is an effective treatment for a drug-resistant patient, an epileptic spike is a widely accepted typical biomarker to identify an epileptogenic zone, and therefore, epileptic spike analysis may be used for preoperative evaluation of the patient with the epilepsy.

### SUMMARY

According to a first aspect of the embodiments of the present application, a method for detecting an epileptic spike is provided, including: obtaining, by a first module of a network model, a local feature of data to be detected, and obtaining, by a second module of the network model, a global feature of the data to be detected; and determining, by a third module of the network model, a detection result of whether there is the epileptic spike in the data to be detected according to the local feature and the global feature. The data to be detected contains a temporal domain and a spatial domain represented by multiple channels, the local feature is a single channel feature, and the global feature is a multichannel feature.

In some embodiments, the method further includes: segmenting the data to be detected to obtain a plurality of multichannel data epochs with a specified time width and a specified number of channels, and each multichannel data epoch of the plurality of multichannel data epochs including a plurality of single-channel data epochs with the specified time width.

In some embodiments, the obtaining, by a first module of a network model, a local feature of data to be detected includes: obtaining, by the first module, a first local feature of each of the plurality of single-channel data epochs; and sequentially combining, by the first module, the first local feature of each of the plurality of single-channel data epochs according to a position of each channel of the multiple channels to obtain the local feature of the multichannel data epoch.

In some embodiments, the obtaining, by a second module of the network model, a global feature of the data to be detected includes: obtaining, by the second module, the global feature of the multichannel data epoch.

In some embodiments, the determining, by a third module of the network model, a detection result of whether there is the epileptic spike in the data to be detected according to the local feature and the global feature includes: inputting the local feature and the global feature into the third module for weighted combination to obtain a combining feature; and outputting, by the third module, the detection result of whether there is the epileptic spike in the multichannel data epoch according to the combining feature.

In some embodiments, the segmenting the data to be detected to obtain a plurality of multichannel data epochs with a specified time width and a specified number of channels includes: as for the temporal domain, segmenting, by using the specified time width corresponding to duration of the epileptic spike, the data to be detected into a plurality of data epochs with the specified time width; and as for the spatial domain, dividing, by using the specified number of channels, the plurality of data epochs according to an anatomical structure of a brain to obtain the plurality of multichannel data epochs.

In some embodiments, two adjacent multichannel data epochs in the plurality of multichannel data epochs overlap each other at a specified time interval.

In some embodiments, the method further includes: determining, by a peak detection model, a peak detection result at a peak of the epileptic spike in the multichannel data epoch according to the detection result; and determining, by a source localization algorithm, a localization of the epileptic spike in a brain according to the peak detection result at the peak of the epileptic spike. The peak detection model includes a first peak detection module, a second peak detection module and a third peak detection module, the first peak detection module is used to output the single channel feature of the multichannel data epoch, the second peak detection module is used to output the multichannel feature of the multichannel data epoch, and the third peak detection module is used to output the peak detection result at the peak of the epileptic spike in the multichannel data epoch according to the single channel feature and the multichannel feature.

In some embodiments, the method further includes: preprocessing raw data to obtain the data to be detected, and the preprocessing includes removing artifacts and normalizing the raw data.

In some embodiments, the removing artifacts includes at least one of the followings: removing noises; correcting a baseline drift of the raw data; and removing electrooculogram and electrocardiograph artifacts.

In some embodiments, the data to be detected includes electroencephalogram (EEG) and magnetoencephalography (MEG) signals.

According to a second aspect of the embodiments of the present application, a method for training a network model is provided, including: obtaining sample data, the sample data including a label with an epileptic spike and a non-epileptic spike; and training the network model based on the sample data. The network model includes a first module configured to output a local feature of the sample data, a second module configured to output a global feature of the sample data, and a third module configured to output a training result of whether there is the epileptic spike in the sample data according to the local feature and the global feature, and the sample data to be detected contains a temporal domain and a spatial domain represented by multiple channels, the local feature is a single channel feature, and the global feature is a multichannel feature.

In some embodiments, the method further includes: segmenting the sample data to obtain a plurality of multichannel sample data epochs with a specified time width and a specified number of channels, each multichannel sample data epoch of the plurality of multichannel sample data epochs including a plurality of single-channel sample data epochs with the specified time width.

In some embodiments, the training the network model based on the sample data includes: inputting the multichannel sample data epoch into the first module to obtain the local feature, and inputting the multichannel sample data epoch into the second module to obtain the global feature; outputting, by the third module, the training result of whether there is the epileptic spike in the multichannel sample data epoch according to the local feature and the global feature; and updating parameters of the network model according to a difference between the training result and the label.

In some embodiments, the first module includes a one-dimensional convolutional neural network, and the inputting the multichannel sample data epoch into the first module to obtain the local feature includes: obtaining, by the one-dimensional convolutional neural network, a first local feature of each of the plurality of single-channel sample data epochs; and sequentially combining the first local feature of each of the plurality of single-channel sample data epochs according to a position of each channel of the multiple channels to obtain the local feature of the multichannel sample data epoch.

In some embodiments, the second module includes an unshared weight two-dimensional convolutional neural network and a shared weight two-dimensional convolutional neural network, and the inputting the multichannel sample data epoch into the second module to obtain the global feature includes: inputting the multichannel sample data epoch into the unshared weight two-dimensional convolutional neural network to obtain a first global feature of the multichannel sample data epoch; inputting the first global feature of the multichannel sample data epoch into the shared weight two-dimensional convolutional neural network to obtain a second global feature of the multichannel sample data epoch; and obtaining the global feature according to the second global feature.

In some embodiments, the outputting, by the third module, the training result of whether there is the epileptic spike in the multichannel sample data epoch according to the local feature and the global feature includes: performing weighted combination on the local feature and the global feature to obtain a combining feature; and outputting the training result of whether there is the epileptic spike in the multichannel sample data epoch according to the combining feature.

In some embodiments, the segmenting the sample data to obtain a plurality of multichannel sample data epochs includes: as for the temporal domain, segmenting, by using the specified time width corresponding to duration of the epileptic spike, the sample data into a plurality of sample data epochs with the specified time width; and as for the spatial domain, dividing, by using the specified number of channels, the plurality of sample data epochs according to an anatomical structure of a brain to obtain the plurality of multichannel sample data epochs.

In some embodiments, two adjacent multichannel sample data epochs in the plurality of multichannel sample data epochs overlap each other at a specified time interval.

In some embodiments, a loss function of the network model is a binary cross entropy loss function, and the updating parameters of the network model according to a difference between the training result and the label includes: obtaining a loss function value according to the binary cross entropy loss function and the difference between the training result and the label; and updating the parameters of the first module, the second module and the third module according to the loss function value.

In some embodiments, the method further includes: preprocessing raw sample data to obtain the sample data, and the preprocessing includes removing artifacts and normalizing the raw data.

In some embodiments, the removing artifacts includes at least one of the followings: removing noises; correcting a baseline drift of the raw data; and removing electrooculogram and electrocardiograph artifacts.

According to a third aspect of the embodiments of the present application, a computer device is provided, including: a memory, a processor and a computer program stored on the memory and executed by the processor. When the processor executes the computer program, the processor performs a method for detecting an epileptic spike as described above and/or a method for training a network model as described above.

According to a fourth aspect of the embodiments of the present application, a computer readable storage medium is provided, on which a computer program is stored. When the computer program is executed by a processor, the processor performs a method for detecting an epileptic spike as described above and/or a method for training a network model as described above.

### BRIEF DESCRIPTION OF DRAWINGS

The above and other objectives, features and advantages of the present application will become more apparent from the more detailed description of the embodiments of the present application taken with reference to the accompanying drawings. The accompanying drawings are used to provide further understanding of the embodiments of the present application, and constitute a part of this specification and explain the present application together with the embodiments of the present application, rather than limiting the present application. Throughout the accompanying drawings, the same reference numerals generally represent the same part or step.

FIG. 1 is a schematic diagram of an implementation scenario according to an embodiment of the present application.

FIG. 2 is a schematic flowchart of a method for detecting an epileptic spike according to an embodiment of the present application.

FIG. 3 is a diagram of MEG data according to an embodiment of the present application.

FIG. 4 is a schematic flowchart of a method for detecting an epileptic spike according to another embodiment of the present application.

FIG. 5 is a diagram of a multichannel data epoch of MEG data according to an embodiment of the present application.

FIG. 6 is a schematic flowchart of a method for detecting an epileptic spike according to still another embodiment of the present application.

FIG. 7 is a visualized graph of a training objective of a peak detection model according to an embodiment of the present application.

FIG. 8a is a visualized graph of a localization result of an epileptic spike at the position of axial plane in a brain according to an embodiment of the present application.

FIG. 8b is a visualized graph of a localization result of an epileptic spike at the position of coronal plane in a brain according to an embodiment of the present application.

FIG. 9 is a schematic flowchart of a method for training a network model according to an embodiment of the present application.

FIG. 10 is a schematic flowchart of a method for training a network model according to another embodiment of the present application.

FIG. 11 is a schematic structural diagram of a network model according to an embodiment of the present application.

FIG. 12 is a schematic flowchart of a method for training a network model according to still another embodiment of the present application.

FIG. 13 is a block diagram of a computer device according to an embodiment of the present application.

### DETAILED DESCRIPTION OF THE EMBODIMENTS

A clear and complete description of technical solutions of the embodiments of the present application will be given below, in combination with the accompanying drawings in the embodiments of the present application. Apparently, the embodiments described below are a part, but not all, of the embodiments of the present application. All of other embodiments, obtained by those skilled in the art based on the embodiments of the present application without any inventive efforts, fall into the protection scope of the present application.

Magnetoencephalography (MEG) is a powerful technology used to detect an interictal epileptic spike noninvasively. A modern MEG system, such as Elekta-Neuromag, with more than 300 sensor channels covering the entire brain, has high spatial resolution for localizing an epileptic spike (i.e., an epileptic discharge). Compared with conventional scalp EEG, MEG recordings are superior since they are immune to signal distortions caused by complex layering of head tissues, so MEG signals have a higher signal-to-noise ratio, and the epileptic spike (i.e., the MEG spike) that appears to be sharper may be more clearly distinguished from background activities. However, the most common clinical practice for detecting the epileptic spike is visual scanning of the MEG signals by experienced neurophysiologists. The vast amount of the MEG recordings with a submillisecond temporal resolution and high number of MEG sensors makes a subjective review process time consuming, and manual spike identification and marking between different spike readers are often inconsistent and not exhaustive. Based on a survey performed in several major hospitals worldwide, a typical neurophysiologist usually takes about 1-3 hours (dependents on clinical experience and complexity of patients' conditions) to screen out the epileptic spike from 90 min MEG recordings with 1000 Hz sample rate per patient. Therefore, an automatic and efficient method for accurately detecting the epileptic spike is much needed in the clinical practice.

Currently, several automatic and effective methods for detecting the epileptic spike have been proposed to detect the epileptic spike in epileptic brain activities for accurate and timely clinical evaluation. In fact, most of these methods have been first applied to the EEG field and then extended to the MEG field. Published methods used for detecting the epileptic spike in interictal MEG data were developed to mainly concentrate on a time feature, a frequency feature, and a wavelet-domain feature. Given a signal segment, a spike detector may implement feature extraction and classification of the epileptic spike and non-epileptic spike. Some methods focus on an amplitude threshold feature of the spike signal (i.e., temporal domain feature extraction), and apply DTW (Dynamic Time Warping) as a distance measure to the detection of the epileptic MEG spike without a priori knowledge of spike template. In another study, the researchers perform automatic detection for the epileptic MEG spike in two steps: performing a subspace decomposition of the MEG signals using a common spatial patterns algorithm for feature extraction and then using LDA (Linear Discriminant Analysis) to classify the epileptic MEG spike. A recent work uses eight statistical values (median, interquartile range, kurtosis, etc.) as features and performs genetic programming with the K-nearest neighbor algorithm for the classification of the interictal epileptic spike.

In summary, the methods described above rely on the priori knowledge about the signals and events, therefore predefining hand-engineered features by indirect signal transformations or mathematical operations. However, routine detection of the MEG signals in the clinical practice is implemented by visual inspection, generally better than any method presented so far. Actually, features of a multivariate time course used in the visual inspection by the experienced neurophysiologists are very difficult. Thus, it is quite desirable to determine feature information of the epileptic spike from raw data directly and effectively without an explicit recipe for a particular feature.

In order to overcome the above-mentioned shortcomings of the existing traditional epileptic spike detection technologies, a method for detecting an epileptic spike based on deep learning is provided in the present application, which can accurately and effectively identify the epileptic spike in the raw data, so as to effectively assist the clinician's preoperative evaluation of epilepsy surgery.

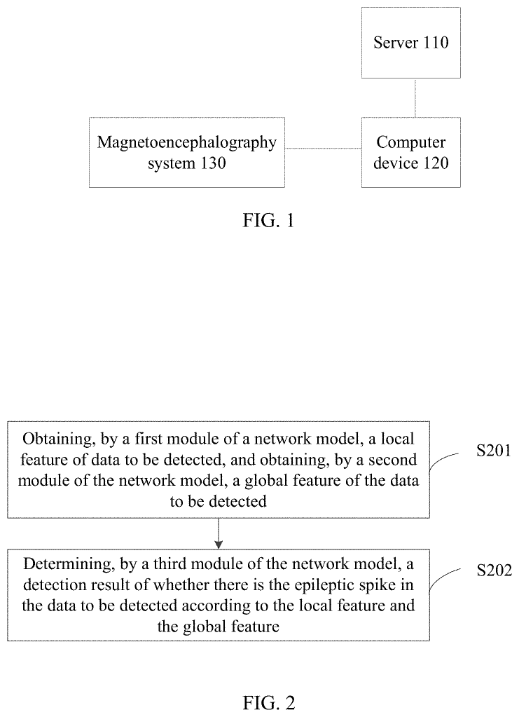

As shown in FIG. 1, an implementation scenario of the present application includes a server 110, a computer device 120 and a Magnetoencephalography system 130, such as the Elekta-Neuromag. The computer device 120 may receive data to be detected from the Magnetoencephalography system 130 used to record the MEG data, and the computer device 120 may also be connected to the server 110 through a communication network. Optionally, the communication network is a wired network or a wireless network.

The data to be detected may be the MEG data obtained by the Magnetoencephalography system 130, but the type of the data to be detected is not limited thereto, the data to be detected may also be EEG data which is obtained from an EEG system. For the convenience of description, the MEG data is used as an example to describe the embodiments of the present application in detail.

The computer device 120 may be a general-purpose computer or a computer device composed of a special integrated circuit, etc., which is not limited in the embodiments of the present application. For example, the computer device 120 may be a mobile terminal device such as a tablet computer, or a personal computer (PC), such as a laptop computer, a desktop computer, etc. Those skilled in the art may know that the number of the above-mentioned computer devices 120 may be one or more, and the types may be the same or different. For example, the above-mentioned computer device 120 may be one, or the above-mentioned computer device 120 may be dozens or hundreds, or more. The embodiments of the present application do not limit the number and type of the computer device 120.

The computer device 120 may be deployed with a network model for detecting the epileptic spike in the MEG data. In some preferred embodiments, the computer device 120 may use the network model to obtain a local feature of the MEG data and a global feature of the MEG data, and then to determine whether there is the epileptic spike in the MEG data according to the local feature and the global feature, which can accurately and effectively identify the epileptic spike in the MEG data, so as to effectively assist the clinician's preoperative evaluation of the epilepsy surgery. The server 110 is one server, or consists of several servers, or a virtualization platform, or a cloud computing service center. In some preferred embodiments, the server 110 trains the network model according to the MEG data which is labeled to distinguish the epileptic spike and the non-epileptic spike. Then the computer device 120 may send the MEG data to the server 110, and the server 110 uses the network model to obtain a local feature of the MEG data and a global feature of the MEG data, and then determine a detection result of whether there is the epileptic spike in the MEG data according to the local feature and the global feature. Finally, the server 110 sends the detection result to the computer device 120 for checking, which can accurately and effectively identify the epileptic spike in the MEG data, so as to effectively assist the clinician's preoperative evaluation of the epilepsy surgery.

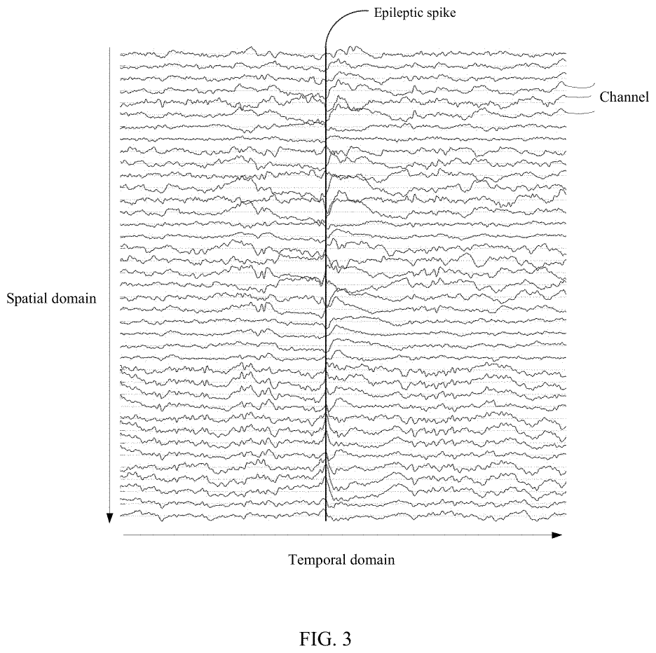

FIG. 2 is a schematic flowchart of a method for detecting an epileptic spike according to an embodiment of the present application. FIG. 3 is a diagram of MEG data according to an embodiment of the present application. The method described in FIG. 2 is performed by a computer device (i.e., a server), but the embodiments of the present application is not limited thereto. The server may be one server, or composed of several servers, or a virtualization platform, or a cloud computing service center, which is not limited in the embodiments of the present application. As shown in FIG. 2, the method includes the following contents.

S201: obtaining, by a first module of a network model, a local feature of data to be detected, and obtaining, by a second module of the network model, a global feature of the data to be detected.

In an embodiment, the data to be detected may be the MEG data, but the type of the data to be detected is not limited thereto, the data to be detected may also be the EEG data. At the same time, the embodiments of the present application do not limit the specific form of the data to be detected, which may be complete data or data after segmentation. For the convenience of description, the MEG data is used as an example to describe the embodiments of the present application in detail.

In an embodiment, during a detection process, the first module and the second module of the network model are processed in parallel, i.e., the first module and the second module are two parallel branches of the network model. After the MEG data is input into the network model, the first module and the second module may process the MEG data in parallel, the first module outputs the local feature, and the second module outputs the global feature.

In an embodiment, as mentioned above, the modern MEG system, such as the Elekta-Neuromag, has more than 300 sensor channels covering the entire brain. As shown in FIG. 3, the MEG data may be a MEG waveform data containing a temporal domain and a spatial domain represented by multiple channels, the temporal domain corresponds to time (i.e., a horizontal direction), and the spatial domain corresponds to the multiple channels of the sensors (i.e., a vertical direction), and a position of a vertical line in FIG. 3 is where the epileptic spike occurs, the epileptic spike in the present application may refer to the interictal epileptic spike. The MEG waveform data is actually composed of multiple waveforms that change with time, i.e., one channel corresponds to one waveform, and the multiple channels correspond to the multiple waveforms.

In an embodiment, the local feature is a single channel feature, i.e., the first module performs single-channel representation learning to obtain a feature of a corresponding channel. Each channel corresponds to one feature, the multiple channels correspond to multiple unrelated features, and therefore, the local feature is defined by the multiple unrelated features.

In an embodiment, the global feature is a multichannel feature, i.e., the second module performs multichannel representation learning to obtain the global feature, and the global feature has an intercorrelation of adjacent channels.

S202: determining, by a third module of the network model, a detection result of whether there is the epileptic spike in the data to be detected according to the local feature and the global feature.

In an embodiment, the third module is used to perform weighted combination on the local feature and the global feature, and output the detection result of whether there is the epileptic spike in the MEG data.

In an embodiment, the detection result may be used to indicate whether there is the epileptic spike in the MEG data, which is not limited in the embodiments of the present application, and the detection result may also be used to indicate at what time there is the epileptic spike.

To sum up, by obtaining the local feature of the MEG data, obtaining the global feature of the MEG data, and determining the detection result of whether there is the epileptic spike in the MEG data according to the local feature and the global feature, detection efficiency and detection accuracy can be increased. Due to automatic feature extraction for the local feature of each channel and the global feature of the multiple channels in the MEG data at the same time, an unreliable problem of relying only on single channel can be avoided, and the problems of traditional epileptic spike detection, such as large calculation, slow detection speed, and low detection accuracy, are solved.

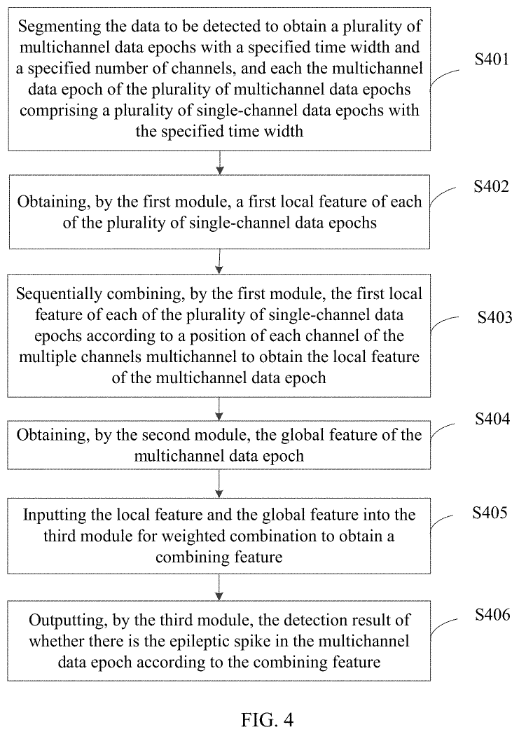

FIG. 4 is a schematic flowchart of a method for detecting an epileptic spike according to another embodiment of the present application. The method is an example of the method as described in FIG. 2. As shown in FIG. 4, the method includes the following contents.

S401: segmenting the data to be detected to obtain a plurality of multichannel data epochs with a specified time width and a specified number of channels, and each multichannel data epoch of the plurality of multichannel data epochs including a plurality of single-channel data epochs with the specified time width.

The MEG data may be directly input into the network model for detecting the epileptic spike, and the MEG data may also be first segmented to generate the plurality of multichannel data epochs, and then the plurality of multichannel data epochs are input into the network model for detecting the epileptic spike, which can reduce the computational load of the network model, so as to increase the detection efficiency of the network model.

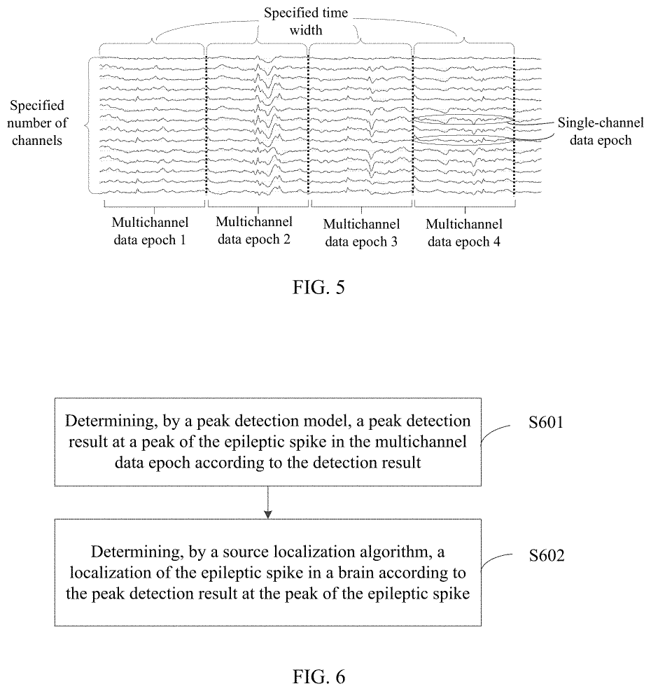

In an embodiment, as shown in FIG. 5, the MEG data is segmented into the plurality of multichannel data epochs, such as a multichannel data epoch 1, a multichannel data epoch 2, a multichannel data epoch 3 and a multichannel data epoch 4. The multichannel data epoch has the specified time width and the specified number of channels, for example, the specified time width is 300 ms, and the specified number of channels is 39 channels. However, the embodiments of the present application do not limit the specific values of the specific time width and the specific number of channels, and those skilled in the art may set the specific values according to specific application requirements.

Referring to FIG. 5, The multichannel data epoch includes a plurality of single-channel data epochs with the specified time width, i.e., the multichannel data epoch may be divided into the plurality of single-channel data epochs with the specified time width, one single-channel data epoch corresponds to one waveform that changes with time in the MEG waveform data, and one multichannel data epoch corresponds to multiple waveforms in the MEG waveform data.

S402: obtaining, by the first module, a first local feature of each of the plurality of single-channel data epochs; and

S403: sequentially combining, by the first module, the first local feature of each of the plurality of single-channel data epochs according to a position of each channel of the multiple channels to obtain the local feature of the multichannel data epoch.

Each of the plurality of single-channel data epochs is input into the first module of the network model, so as to obtain the first local feature of each single-channel data epoch, i.e., the first module performs the single-channel representation learning to obtain the first local feature of each single-channel data epoch.

When the first local feature of each single-channel data epoch is obtained, the first local feature of each single-channel data epoch is sequentially combined by the first module according to a position of each channel of the multiple channels, so as to obtain the local feature of the multichannel data epoch, i.e., in the MEG data, there is a specific location relationship between the multiple channels, the first local feature of each single-channel data epoch is sequentially combined according to the specific location relationship, so as to obtain the local feature of the multichannel data epoch.

One first local feature corresponds to one single-channel data epoch in one multichannel data epoch, and therefore, the plurality of single-channel data epochs in said multichannel data epoch correspond to a plurality of first local features. One local feature obtained by sequentially combining the plurality of first local features corresponds to one multichannel data epoch, and therefore, the plurality of multichannel data epochs in the MEG data correspond to a plurality of local features, and each of the plurality of local features contains the plurality of first local features.

S404: obtaining, by the second module, the global feature of the multichannel data epoch.

Each multichannel data epoch in the plurality of multichannel data epochs is input into the second module, so as to obtain the global feature of the multichannel data epoch, i.e., the second module performs the multichannel representation learning to obtain the global feature of the multichannel data epoch, and the global feature has the intercorrelation of the adjacent channels.

One multichannel data epoch corresponds to one global feature, and therefore, the plurality of multichannel data epochs in the MEG data correspond to a plurality of global features. As mentioned above, the plurality of multichannel data epochs in the MEG data correspond to the plurality of local features, and therefore, a same multichannel data epoch in MEG data corresponds to one local feature and one global feature at the same time.

S405: inputting the local feature and the global feature into the third module for weighted combination to obtain a combining feature.

The local feature and the global feature are input into the third module of the network model for the weighted combination, so as to obtain the combining feature.

S406: outputting, by the third module, the detection result of whether there is the epileptic spike in the multichannel data epoch according to the combining feature.

The detection result of whether there is the epileptic spike in the multichannel data epoch is output by the third module according to the combining feature.

Thus, by inputting the MEG data into the network model, the detection result of whether there is the epileptic spike in the multichannel data epoch is obtained, and each multichannel data epoch may get a corresponding detection result.

In an embodiment of the present application, the segmenting the data to be detected to obtain a plurality of multichannel data epochs with a specified time width and a specified number of channels includes: as for the temporal domain, segmenting, by using the specified time width corresponding to duration of the epileptic spike, the data to be detected into a plurality of data epochs with the specified time width; and as for the spatial domain, dividing, by using the specified number of channels, the plurality of data epochs according to an anatomical structure of a brain to obtain the plurality of multichannel data epochs.

As for the temporal domain, the MEG data may be segmented into the plurality of data epochs with the specified time width by using the specified time width corresponding to duration of the epileptic spike. The data epoch refers to as an epoch that only segments the time by using the specified time width, and the specified time width is determined by considering the duration of the epileptic spike, i.e., the specified time width should be greater than or equal to the duration of the epileptic spike, so as to make the data epoch contain a complete epileptic spike, for example, the specified time width is 300 ms.

As mentioned above, the modern MEG system, such as Elekta-Neuromag, has more than 300 sensor channels (i.e., the channel in the embodiments of the present application) covering the entire brain. As for the spatial domain, the data epoch is then divided into the multichannel data epoch by using the specified number of channels according to the anatomical structure of the brain. The multichannel data epoch refers to as an epoch that not only segments the time by using the specified time width, but also divides the channels by using the specified number of channels according to the anatomical structure of the brain.

For example, 306 channels are divided into eight nonoverlapping sensor groups based on the anatomical structure of the brain, roughly including the left temporal (LT), right temporal (RT), left parietal (LP), right parietal (RP), left occipital (LO), right occipital (RO), left frontal (LF), and right frontal (RF) areas of the head. Except for the L/RO areas, which cover 36 channels, the rest of the segmented areas cover 39 channels. As for the L/RO areas which cover 36 channels, zero-padding needs to be performed on the epochs from the L/RO areas with 36 channels, so that each area covers 39 channels. Thus, the data to be detected input into the network model may be unified.

However, the embodiments of the present application do not limit the specific division of the brain, i.e., the embodiments of the application do not limit the specific division of the sensor channels.

In an embodiment, the MEG data is segmented into the plurality of multichannel data epochs with 300 ms and 39 channels, i.e., a size of the multichannel data epoch is 39*300. The multichannel data epoch input into the network model for epileptic spike detection may be a matrix with a size of 39*300.

In an embodiment of the present application, two adjacent multichannel data epochs in the plurality of multichannel data epochs overlap each other at a specified time interval.

The epileptic spike is rare in real recordings and approximately 90% of the multichannel data epochs can expected to be non-epileptic spike epochs. Thus, an overlapping area between the two adjacent multichannel data epochs is mainly to prevent the epileptic spike from being divided into two incomplete parts when segmenting the MEG data, resulting in missed detection of the network model.

The multichannel data epoch is segmented into epochs of 300 ms with 50% or other percentage overlapping between two successive epochs, i.e., any two adjacent multichannel data epochs in the plurality of multichannel data epochs overlap each other at the specified time interval of 150 ms or other length of time. However, the specific value of the specified time interval is not limited in the embodiments of the present application, the specified time interval may also set to another value, such as 50 ms.

FIG. 6 is a schematic flowchart of a method for detecting an epileptic spike according to still another embodiment of the present application. As shown in FIG. 6, the method of FIG. 2 or FIG. 4 further includes the following contents.

S601: determining, by a peak detection model, a peak detection result at a peak of the epileptic spike in the multichannel data epoch according to the detection result.

The peak detection model includes a first peak detection module, a second peak detection module and a third peak detection module, the first peak detection module is used to output the single channel feature of the multichannel data epoch, the second peak detection module is used to output the multichannel feature of the multichannel data epoch, and the third peak detection module is used to output the peak detection result at the peak of the epileptic spike in the multichannel data epoch according to the single channel feature and the multichannel feature.

The peak detection result at a peak of the epileptic spike may be understood as peak time of the epileptic spike, i.e., when the epileptic spike occurs, the epileptic spike duration ranges from 20 to 70 ms, and the peak time of the epileptic spike may be found out by using the peak detection model.

S602: determining, by a source localization algorithm, a localization of the epileptic spike in a brain according to the peak detection result at the peak of the epileptic spike. When the detection result of whether there is the epileptic spike in the multichannel data epoch is obtained, the detection result may be used to determine the peak detection result at the peak of the epileptic spike in the multichannel data epoch. Specifically, the multichannel data epoch with the epileptic spike detected by the network model described above may be input into the peak detection model for obtaining the peak detection result at the peak of the epileptic spike in the multichannel data epoch. Then, when the peak detection result at the peak of the epileptic spike is determined, the peak detection result at the peak of the epileptic spike in a corresponding data epoch containing all the channels may be processed by using the source localization algorithm, and the localization of the epileptic spike in the brain may be achieved.

The peak detection model is proposed based on the network model described above. Specifically, training data for training the peak detection model is the same as training data for training the network model described above, and detection data of the peak detection model is the same as detection data of the network model described above, i.e., the training data for training the peak detection model and the detection data for the peak detection model may be the multichannel data epoch obtained by using the method of the step of S401. But ground truth (i.e., training objective) of which are different, the ground truth of the network model described above is a two classification problem, and the ground truth of the peak detection model is cleverly converted into a 300 classification problem, i.e., the ground truth is 300 categories.

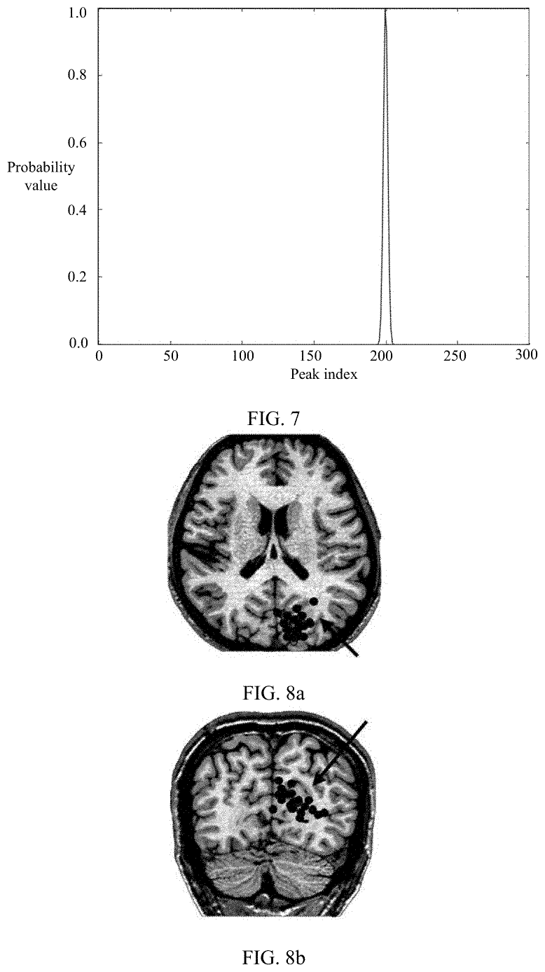

It cannot be simply set as 1 at a peak of the epileptic spike, and 0 at the rest region, i.e., one-hot encoding. However, adopting the one-hot encoding is difficult to train the peak detection model successfully. For the peak of the epileptic spike, the overall process of the peak of the epileptic spike is from rapid rise to peak, and then rapid decline trend, so suppose that a curve of the peak conforms to the normal probability density function distribution. As shown in FIG. 7, as for the multichannel data epoch with the specified time width of 300 ms and the specified number of channels of 39 channels, if it is the peak at 200 ms, the peak at 200 ms is set to 1, and the rest region shows a downward trend according to the normal probability density function. The specific implementation ideas are as follows:

label_index is the index where the peak is located, segment_length is the length of the multichannel data epoch, i.e., 300 ms, the average value is equal to (label_index−segment_length/2)/(segment_length/2), the standard deviation is set to 0.01, and x is equal to segment_length samples evenly spaced and calculated in the interval [−1,1]. The ground truth y of the peak detection model may be obtained by substituting the mean value, standard deviation and x into the normal probability density function.

The first peak detection module of the peak detection model is the same as the first module of the network model described above, the second peak detection module of the peak detection model is the same as the second module of the network model described above, and the third peak detection module of the peak detection model is different from the third module of the network model described above. The third peak detection module of the peak detection model is modified as follows:

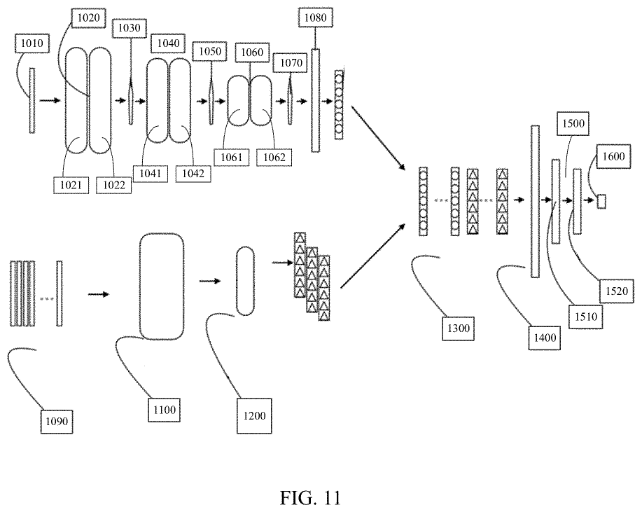

The number of fully connected layers in the third peak detection module is modified, i.e., the cascade fully connected layer 1500 in FIG. 11 described below is modified into one fully connected layer, said fully connected layer includes 2048 layers, and the activation function is leaky relu used to increase the nonlinear fitting ability. An output category is changed from the original two classification to 300 classification, i.e., the output layer may be said fully connected layer using the softmax activation function, which is used to reduce a 1*2048 matrix to a matrix with a length of 300, and the matrix with a length of 300 may represent the probability of the peak of the epileptic spike.

After training the peak detection model is completed, the parameters of the peak detection model and the network structure of the peak detection model are completely saved as an h5 format file. When subsequently used for prediction of new MEG data, it is only necessary to load the format file to predict the multichannel data epoch, and obtain the peak detection result at the peak of the epileptic spike in the multichannel data epoch.

Specifically, FIG. 8a and FIG. 8b show a visualized graph obtained by using a source localization algorithm. Small dots pointed out by the arrow on the visualized graph correspond to a source localization result of the epileptic spike.

However, the embodiments of the present application do not limit the specific type of the source localization algorithm, and the source localization algorithm may also include other suitable methods.

In an embodiment of the present application, the method further includes: preprocessing raw data to obtain the data to be detected, and the preprocessing includes removing artifacts and normalizing the raw data.

Before detecting the epileptic spike, the raw data may be preprocessed to obtain the MEG data to be detected. However, the embodiments of the present application do not specifically limit whether to perform the preprocessing or not, and the raw data may be directly input into the network model for epileptic spike detection.

The preprocessing includes removing artifacts and normalizing the raw data. The embodiments of the present application do not limit the sequence of removing artifacts and normalizing the raw data, and artifacts may be removed first, then the raw data is normalized, or the raw data may be normalized first, and then artifacts is removed.

By normalizing the raw data, the MEG data to be detected input into the network model conforms to a standard normal distribution, so that complexity of the network model learning can be reduced and convergence ability of the network model can be improved.

In an embodiment of the present application, the removing artifacts includes at least one of the followings: removing noises; correcting a baseline drift of the raw data;

and removing electrooculogram and electrocardiograph artifacts.

In an embodiment, a High-pass filter may be used to remove a baseline drift noise and a power frequency interference noise, and a Low-pass filter may be used to remove noise interference from outside.

In an embodiment, the frequency range of 1 Hz˜100 Hz is used for removing the noises, and thus, the interference caused by various noises in the MEG data can be effectively removed, so as to further improve the detection accuracy of the network model.

In an embodiment, an independent component analysis (ICA) algorithm may be used to remove the electrooculogram and electrocardiograph artifacts.

To sum up, by performing the preprocessing, the complexity of the network model learning can be reduced and the convergence ability of the network model can be improved.

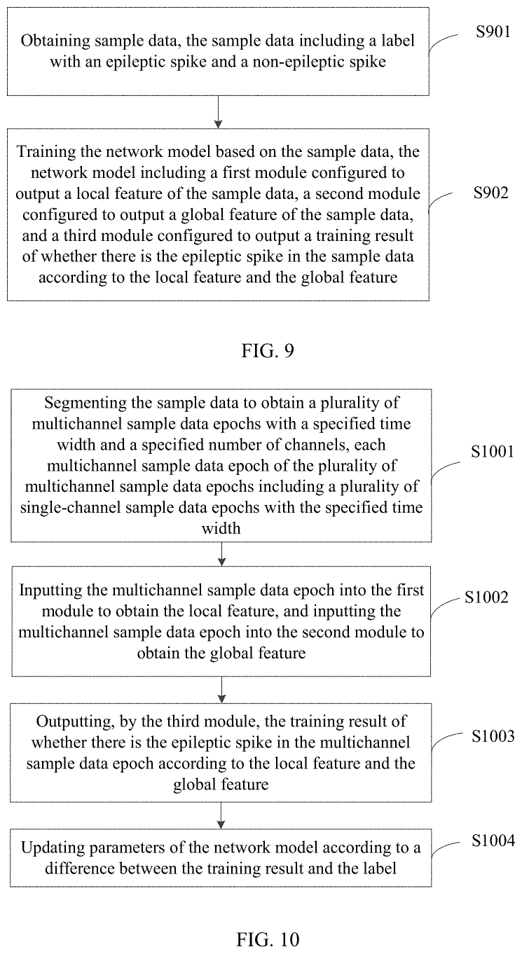

FIG. 9 is a schematic flowchart of a method for training a network model according to an embodiment of the present application. The method described in FIG. 9 is performed by a computer device (i.e., a server), but the embodiments of the present application are not limited to thereto. The server may be one server, or composed of several servers, or a virtualization platform, or a cloud computing service center, which is not limited in the embodiments of the present application. As shown in FIG. 9, the method includes the following contents.

S901: obtaining sample data, the sample data including a label with an epileptic spike and a non-epileptic spike.

The sample data mentioned in this embodiment of the present application belongs to a same type of the data to be detected described in the embodiments of the method for detecting the epileptic spike. The specific explanation of the sample data may not be repeated here. Please refer to the relevant description of the data to be detected in the above embodiments of the present application.

The difference between the sample data described in this embodiment of the present application and the data to be detected described in the embodiments of the method for detecting the epileptic spike is only that the sample data is labeled manually, and therefore, the sample data includes the label with the epileptic spike and the non-epileptic spike.

The sample data may be MEG data, but the type of the sample data is not limited thereto, the sample data may also be EEG data. At the same time, the embodiments of the present application do not limit the specific form of the sample data, which may be complete data or data after segmentation. For the convenience of description, MEG sample data is used as an example to describe the embodiments of the present application in detail.

S902: training the network model based on the sample data.

In an embodiment, the network model includes a first module configured to output a local feature of the sample data, a second module configured to output a global feature of the sample data, and a third module configured to output a training result of whether there is the epileptic spike in the sample data according to the local feature and the global feature.

In an embodiment, as mentioned above, the modern MEG system, such as Elekta-Neuromag, has more than 300 sensor channels covering the entire brain. The MEG sample data may be a MEG waveform data containing a temporal domain and a spatial domain represented by multiple channels, the temporal domain corresponds to time, and the spatial domain corresponds to the multiple channels of the sensors. The MEG waveform data is actually composed of multiple waveforms that change with time, i.e., one channel corresponds to one waveform, and the multiple channels correspond to the multiple waveforms.

In an embodiment, the local feature is a single channel feature, i.e., the first module performs single-channel representation learning to obtain a feature of a corresponding channel. Each channel corresponds to one feature, the multiple channels correspond to multiple unrelated features, and therefore, the local feature is defined by the multiple unrelated features.

In an embodiment, the global feature is a multichannel feature, i.e., the second module performs multichannel representation learning to obtain the global feature, and the global feature has an intercorrelation of adjacent channels.

In an embodiment, the third module is used to perform weighted combination on the local feature and the global feature, and output the detection result of whether there is the epileptic spike in the MEG sample data.

In an embodiment, the detection result may be used to indicate whether there is the epileptic spike in the MEG sample data, which is not limited in the embodiments of the present application, and the detection result may also be used to indicate at what time there is the epileptic spike.

In an embodiment, the first module, the second module and the third module may also refer to deep learning models, such as a convolutional neural network (CNN), a deep neural network (DNN), or a recurrent neural network (RNN), etc., and these networks may be backbone networks such as ResNet, ResNeXt or DenseNet. The embodiments of the present application do not limit the specific types of the first module, the second module and the third module.

To sum up, by obtaining the local feature of the MEG sample data, obtaining the global feature of the MEG sample data, and determining the detection result of whether there is the epileptic spike in the MEG sample data according to the local feature and the global feature, detection efficiency and detection accuracy can be increased. Due to automatic feature extraction for the local feature of each channel and the global feature of the multiple channels in the MEG sample data at the same time, an unreliable problem of relying only on single channel can be avoided.

As shown in FIG. 10, the method of FIG. 9 further includes the following contents.

S1001: segmenting the sample data to obtain a plurality of multichannel sample data epochs with a specified time width and a specified number of channels, each multichannel sample data epoch of the plurality of multichannel sample data epochs including a plurality of single-channel sample data epochs with the specified time width.

The step of S1001 mentioned in this embodiment of the present application is similar to the step of S401 described in the embodiments of the method for detecting the epileptic spike. The specific explanation of the step of S1001 mentioned in this embodiment may not be repeated here. Please refer to the relevant description of the step of S401 described in the embodiments of the method for detecting the epileptic spike.

S1002: inputting the multichannel sample data epoch into the first module to obtain the local feature, and inputting the multichannel sample data epoch into the second module to obtain the global feature.

In an embodiment, during a training process, the first module and the second module of the network model are processed in parallel, i.e., the multichannel sample data epoch is input into the first module and the second module simultaneously. The first module and the second module may process the multichannel sample data epoch in parallel, and then the first module outputs the local feature, and the second module outputs the global feature.

S1003: outputting, by the third module, the training result of whether there is the epileptic spike in the multichannel sample data epoch according to the local feature and the global feature.

The steps of S1002 and S1003 mentioned in this embodiment of the present application are similar to the related steps described in the embodiments of the method for detecting the epileptic spike. The specific explanation of the steps of S1002 and S1003 mentioned in the embodiments of the present application may not be repeated here. Please refer to the relevant description of the related steps described in the embodiments of the method for detecting the epileptic spike.

S1004: updating parameters of the network model according to a difference between the training result and the label.

After training each multichannel sample data epoch with the network model, the corresponding training results may be output, i.e., each multichannel sample data epoch in the plurality of multichannel sample data epochs corresponds to one corresponding training result.

By comparing the training result corresponding to the multichannel sample data epoch with the label on the multichannel sample data epoch, the difference between the two may be obtained, and then the difference is back-propagation to update the parameters of the network model. The above steps are iteratively performed until convergence. At this time, the network model after training is obtained.

In an embodiment of the present application, the first module includes a self-designed network structure with one dimensional convolution layer (i.e., a one-dimensional convolutional neural network) applied to multichannel timing MEG or EEG data (i.e., multichannel sample data epoch), and the inputting the multichannel sample data epoch into the first module to obtain the local feature includes: inputting each of the plurality of single-channel sample data epochs of the multichannel sample data epoch into to obtaining, by the one-dimensional convolutional neural network, a first local feature of each of the plurality of single-channel sample data epochs; and sequentially combining the first local feature of each of the plurality of single-channel sample data epochs according to a position of each channel of the multiple channels to obtain the local feature of the multichannel sample data epoch.

For example, the one-dimensional convolutional neural network may include includes six convolutional layers, four dropout blocks, three max-pooling layers and one dense layer.

As mentioned above, the multichannel data epoch input into the network model for epileptic spike detection may be the matrix with a size of 39*300, and therefore, suppose that the multichannel sample data epoch may be a M*N matrix (M represents the number of channels, and N represents the length of time), and the single-channel sample data epoch is a 1*N matrix.

As shown in FIG. 11, the processing process of each of the plurality of single-channel sample data epochs by the one-dimensional convolutional neural network may be described in detail.

Only when a filter is defined may the one-dimensional convolutional neural network learn a single feature. The 1*N matrix 1010 is input into a first cascade 1D convolution layer 1020 including a first convolution layer 1021 and a second convolution layer 1022 which have a filter (also known as feature detector) with convolution kernel size of K*(1*j), i.e., the number of filters is K and the size of the convolution kernel is (1*j). In this way, K different features are obtained and trained in the first cascade 1D convolution layer 1020, i.e., after the first convolution layer 1021 and the second convolution layer 1022, a (N−2j+2)*K matrix is output.

In order to reduce the output complexity and prevent data over fitting, a first maximum pooling layer 1030 is used after the first cascade 1D convolution layer 1020. For example, the first maximum pooling layer 1030 with a size of 2 is selected, and this means that a size of an output matrix of the first maximum pooling layer 1030 is only half of that of an input matrix of the first maximum pooling layer 1030. Thus, after the (N−2j+2)*K matrix is input into the first maximum pooling layer 1030, a (N/2−j+1)*K matrix is output.

Then, the (N/2−j+1)*K matrix is input into a second cascade 1D convolution layer 1040 including a third convolution layer 1041 and a fourth convolution layer 1042 which have a filter (also known as feature detector) with convolution kernel size of L*(1*i), i.e., the number of filters is L and the size of the convolution kernel is (1*i). In this way, L different features are obtained and trained in the second cascade 1D convolution layer 1040, i.e., after the (N/2−j+1)*K matrix is input into the third convolution layer 1041 and the fourth convolution layer 1042, a (N/2−j−2i+3)*L matrix is output.

A second maximum pooling layer 1050 is used after the second cascade 1D convolution layer 1040. For example, the second maximum pooling layer 405 with a size of 2 is selected, and this means that a size of an output matrix of the second maximum pooling layer 1050 is only half of that of an input matrix of the second maximum pooling layer 1050. Thus, after the (N/2−j−2i+3)*L matrix is input into the second maximum pooling layer 1050, a (N/4−j/2−i+1.5)*L matrix is output.

In order to learn higher-level features, a third cascade 1D convolution layer 1060 is used here, and the third cascade 1D convolution layer 1060 includes a fifth convolution layer 1061 and a sixth convolution layer 1062 which have a filter (also known as feature detector) with convolution kernel size of P*(1*k), i.e., the number of filters is P and the size of the convolution kernel is (1*k). In this way, P different features are obtained and trained in the third cascade 1D convolution layer 1060, i.e., after the (N/4−j/2−i+1.5)*L matrix is input into the fifth convolution layer 1061 and the sixth convolution layer 1062, a (N/4−j/2−i−2k+3.5)*P matrix is output.

To further avoid the occurrence of over fitting, a global maximum pooling layer 1070 is used after the third cascade 1D convolution layer 1060, which expands the (N/4−j/2−i−2k+3.5)*P matrix into a 1*P matrix. Since each column of the matrix output by the above cascade convolution layer contains a weight of one filter of the feature detector, after passing through the global maximum pooling layer 1070, only one weight is left for each feature detector.

In order to increase the ability of nonlinear fitting, a fully connected layer 1080 is used after the global maximum pooling layer 1070, which also outputs a 1*P matrix.

As mentioned above, the value range of N is greater than 100; the value range of K is greater than 0; the value range of P is greater than 0; the value range of j is greater than 0, and (M−2j+2) is greater than 0; the value range of i is greater than 0, and (N/2−j−2i+3) is greater than 0; the value range of K is greater than 0, and (N/4−j/2−i−2k+3.5) is greater than 0; and the value range of L is greater than 0.

In the above process, the first local feature of each of the plurality of single-channel sample data epochs is obtained, i.e., one 1*P matrix corresponds to one first local feature. And then, the first local features corresponding to the plurality of single-channel sample data epochs are sequentially combined together according to the position of each channel of the multiple channels, i.e., a plurality of 1*P matrices are sequentially combined together to obtain the local feature of the multichannel sample data epoch. Since the multichannel sample data epoch may be the M*N matrix, i.e., the number of the multiple channels is M, the local feature corresponds to an M*P matrix.

Specifically, each convolutional layer mainly includes two execution steps successively: a convolution operation with multiple convolution kernels and performing activation function with a leaky rectified linear unit (leaky relu), which are denoted as:

z^{[l+1]}=W^{[l+1]}x^{[l]}+b^{[l+1]} (1)

x^{[l+1]}=g(z^{[l+1]}) (2)

In Eq. (1), the activations x^{[l]} from layer l act as the input for layer l+1, b^{[l+1]} is a bias vector, and W^{[l+1]} is a weight matrix. The formula of the activation function leaky relu in Eq. (2) is g(z)=max (αz, z), with commonly used gradient value α=0.1. The activations x^{[l+1]} may be obtained by Eq. (2). Pooling may be performed after each convolution layer to downsample the inputs using max operation (pooling size=2). The last activations x^{[end]} act as the input for the next dense layer (i.e., fully connected layers), and then the output of the last dense layer would be calculated by a logistic sigmoid activation function of g(z)=1/(1+e^{-z}). This probability-like output of the sigmoid function permits only values between 0 and 1 due to characteristics of function itself, that is, a lower value indicates the single-channel sample data epoch is less likely to be the epileptic spike; in contrast, a higher value means the single-channel sample data epoch has high probability to be the epileptic spike.

The first local feature hⁱ for a particular channel may be obtained from the output of the activation function. The first module using a sequential combination for the multichannel sample data epoch may be expressed by:

h_{local}=[h₁, h₂, . . . hᵢ, . . . h_{M}], M=39 (3)

In Eq. (3), M is the number of the channels for the multichannel sample data epoch.

In an embodiment of the present application, the second module includes a self-designed network structure combining a shared convolution layer and an unshared convolution layer (i.e., an unshared weight two-dimensional convolutional neural network and a shared weight two-dimensional convolutional neural network) applied to multichannel timing MEG or EEG data (i.e., multichannel sample data epoch), and the inputting the multichannel sample data epoch into the second module to obtain the global feature includes: inputting the multichannel sample data epoch into the unshared weight two-dimensional convolutional neural network to obtain a first global feature of the multichannel sample data epoch; inputting the first global feature of the multichannel sample data epoch into the shared weight two-dimensional convolutional neural network to obtain a second global feature of the multichannel sample data epoch; and obtaining the global feature according to the second global feature.

As mentioned above, suppose that the multichannel sample data epoch may be the M*N matrix (M represents the number of channels, and N represents the length of time). As shown in FIG. 11, the processing process of multichannel sample data epoch by the unshared weight two-dimensional convolutional neural network and the shared weight two-dimensional convolutional neural network may be described in detail.

The unshared weight two-dimensional convolutional neural network includes an unshared weight two-dimensional convolutional layer 1100. Only when a filter is defined may the unshared weight two-dimensional convolutional neural network learn a single feature. The M*N matrix 1090 is input into the unshared weight two-dimensional convolutional layer 1100 having a filter (also known as feature detector) with convolution kernel size of K′*(i′*j′), i.e., the number of filters is K′ and the size of the convolution kernel is (i′*j′). In this way, K′ different features are obtained and trained in the unshared weight two-dimensional convolutional layer 1100, i.e., after the unshared weight two-dimensional convolutional layer 1100, a (M−j′+1)*(N−i′+1)*K′ matrix is output.

The shared weight two-dimensional convolutional neural network includes a shared weight two-dimensional convolutional layer 1200. The (M−j′+1)*(N−i′+1)*K′ matrix is input into the shared weight two-dimensional convolutional layer 1200 having a filter with convolution kernel size of L′*(1*1), i.e., the number of filters is L′ and the size of the convolution kernel is (1*1). In this way, L′ different features are obtained and trained in the shared weight two-dimensional convolutional layer 1200, i.e., after the (M−j′+1)*(N−i′+1)*K′ matrix is input into the shared weight two-dimensional convolutional layer 1200, a (M−j′+1)*(N−i′+1)*L′ matrix is output.

The first global feature of the multichannel sample data epoch corresponds to the (M−j′+1)*(N−i′+1)*K′ matrix, and the second global feature of the multichannel sample data epoch corresponds to the (M−j′+1)*(N−i′+1)*L′ matrix.

The obtaining the global feature according to the second global feature includes performing reshape operation on the second global feature of the multichannel sample data epoch to obtain the global feature of the multichannel sample data epoch. Specifically, the reshape operation is performed on the (M−j′+1)*(N−i′+1)*L′ matrix, so as to output a (M−j′+1)*L′ matrix. The global feature of the multichannel sample data epoch corresponds to the (M−j′+1)*L′ matrix.

As mentioned above, the value range of K′ is greater than 0; the value range of j′ is greater than 0, and the value range of j′ is less than M; the value range of i′ is greater than 0, and the value range of i′ is equal to N; and the value range of L′ is greater than 0.

At the same time, the size of P in the one-dimensional convolutional neural network is consistent with that of L′ in the shared weight two-dimensional convolutional neural network, so as to facilitate the weighted combination of the following third modules.

Specifically, the unshared weight two-dimensional convolutional neural network and the shared weight two-dimensional convolutional neural network consist of a series of 2D convolution with its filters, which is denoted as:

h_{global}=g(W_{g}x_{1:M}+b_{g}) (4)

In Eq. (4), h_{global} represents global-based (multiple channels) representation learning, and x_{1:M}=(x₁, x₂, . . . , x_{M}) represents cluster-based segmented sensor waveforms. Eq. (4) sufficiently considers the characteristics of multichannel cooccurrences when the epileptic spike occurs. The relevant channel information could be captured sequentially through two kinds of convolution layers: the unshared weight two-dimensional convolutional neural network (i.e., an unshared weight 2D locally-connected layer) which has K′ filters with size of i′*j′ and stride set to 1, as well as the shared weight two-dimensional convolutional neural network (i.e., a shared weight 2D convolution layer) which has L′ filters with size of 1×1 and stride 1.

An unshared weight of the unshared weight two-dimensional convolutional neural network means weights of filters are unshared, i.e., a different set of filters is applied at different channels of the multichannel sample data epoch. A shared weight of the shared weight two-dimensional convolutional neural network means weights of filters are shared, i.e., a same set of filters is applied at the multiple channels of the multichannel sample data epoch.

When extracting the global feature, it is necessary for each channel not to share the weight, i.e., different network weights are used to extract features of different channels, so the shared weight may not affect feature extraction effect of each channel. Therefore, the unshared weight two-dimensional convolutional neural network is adopted first, and then the feature of each channel need to be fused, and therefore, the shared weight two-dimensional convolutional neural network is adopted, and finally the global feature to be fused is obtained.

In fact, in the process of obtaining the first global feature, each different channel of the multichannel sample data epoch is applied with a different set of filters. In other words, different weight value is applied to each different cluster-based segmented sensor waveform corresponding to each different channel in the multichannel sample data epoch, so as to obtain the (M−j′+1)*(N−i′+1)*K′ matrix (i.e., the first global feature).

In the process of obtaining the second global feature, the multiple channels of the multichannel sample data epoch is applied with a same set of filters. In other words, a same weight value is applied to each cluster-based segmented sensor waveform corresponding to each channel in the multichannel sample data epoch, so as to obtain the (M−j′+1)*(N−i′+1)*L′ matrix (i.e., the second global feature).

In an embodiment of the present application, the outputting, by the third module, the training result of whether there is the epileptic spike in the multichannel sample data epoch according to the local feature and the global feature includes: performing weighted combination on the local feature and the global feature to obtain a combining feature; and outputting the training result of whether there is the epileptic spike in the multichannel sample data epoch according to the combining feature.

As shown in FIG. 11, the weighted combination 1300 is performed on the M*P matrix corresponding to the local feature and the (M−j′+1)*L′ matrix corresponding to the global feature, and since the size of P in the one-dimensional convolutional neural network is consistent with that of L′ in the shared weight two-dimensional convolutional neural network, a (2M−j′+1)*L′ matrix is obtained. The (2M−j′+1)*L′ matrix corresponds to the combining feature.

For example, the third module may also include a flattening layer 1400, a cascade fully connected layer 1500 including a first fully connected layer 1510 and a second fully connected layer 1520, and an output layer 1600.

The flattening layer 413 is used to perform a flattening operation on the (2M−j′+1)*L′ matrix (i.e., the combining feature), so as to obtain a 1*[(2M−j′+1)*L′] matrix.

In order to increase the ability of nonlinear fitting, the first fully connected layer 1510 includes 512 layers, the second fully connected layer 1520 includes 64 layers, and the activation functions used by all layers are relu. The 1*[(2M−j′+1)*L′] matrix is input into the first fully connected layer 1510 and the second fully connected layer 1520, and then a 1*64 matrix is output.

The output layer 1600 may be a fully connected layer using the logistic sigmoid activation function, which is used to reduce the 1*64 matrix to a matrix with a length of 1, and the matrix with a length of 1 may represent the probability of the occurrence of the epileptic spike, i.e., the training result of whether there is the epileptic spike in the multichannel sample data epoch.

Specifically, the weighted combination is performed on the local feature and the global feature to detect the epileptic spike:, which is denoted as:

h_{combi}=g(W_{combi}[w₁h_{local}+w₂h_{global}]+b_{combi}) (5)

In Eq. (5), w₁ and w₂ are constant weight values. Note that W and b in Eq. (4) and Eq. (5) are the weight matrix and bias vector, respectively.

However, it should be understood that the specific structures of the first module, the second module, and the third module described above are only an example of this application, and other structures that conform to the design concept of this application are applicable to this application.

In an embodiment of the present application, the segmenting the sample data to obtain a plurality of multichannel sample data epochs includes: for a temporal domain, segmenting, by using the specified time width corresponding to duration of the epileptic spike, the sample data into a plurality of sample data epochs with the specified time width; and for a spatial domain, dividing, by using the specified number of channels, the plurality of sample data epochs according to an anatomical structure of a brain to obtain the plurality of multichannel sample data epochs.

This step of this embodiment is the same as the relevant step described in the embodiments of the method for detecting the epileptic spike. The specific explanation of this step may not be repeated here. Please refer to the relevant description of the relevant step described in the embodiments of the method for detecting the epileptic spike.

In an embodiment of the present application, two adjacent multichannel sample data epochs in the plurality of multichannel sample data epochs overlap each other at a specified time interval.

This embodiment is the same as the relevant embodiments of the method for detecting the epileptic spike. The specific explanation of this embodiment may not be repeated here. Please refer to the relevant description of the relevant embodiments of the method for detecting the epileptic spike.

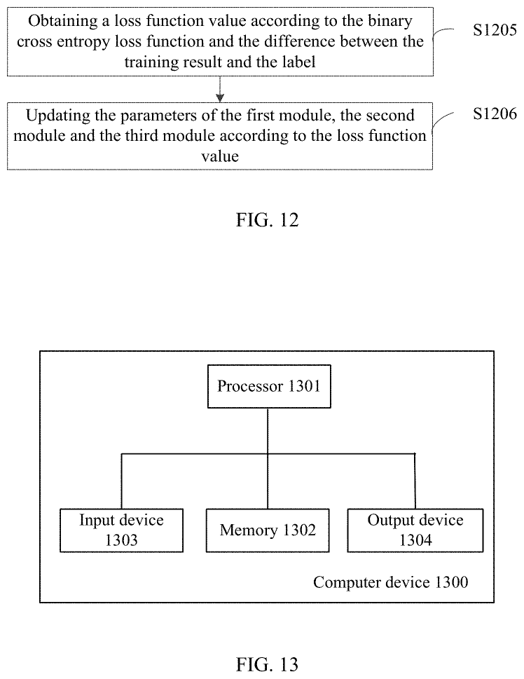

FIG. 12 is a schematic flowchart of a method for training a network model according to still another embodiment of the present application. As shown in FIG. 12, the method of FIG. 10 further includes the following contents. I.e., when a loss function of the network model is a binary cross entropy loss function, and the updating parameters of the network model according to a difference between the training result and the label includes following contents.

S1205: obtaining a loss function value according to the binary cross entropy loss function and the difference between the training result and the label.

S1206: updating the parameters of the first module, the second module and the third module according to the loss function value.

In an embodiment, an optimization function used in a training process of the network model is Adam, a learning rate is set to 2e−4, the binary cross entropy loss function is used as the loss function, and the batch size of the training is 64.

An early stopping technology is adopted in the training process to prevent over fitting. As the number of training rounds increases, if an error is found on a verification set, the training is stopped. The weight after the stop is used as the final parameter of the network model.

The loss function value is obtained according to the binary cross entropy loss function and the difference between the training result and the label. The parameters of the first module, the second module and the third module may be updated according to the loss function value. The smaller the loss function value is, the closer the training result is to the label, and the higher the accuracy of the network model is. Conversely, the larger the loss function value, the lower the accuracy of the network model.

The binary cross entropy loss function is usually used for solving the problem of slow update and convergence speed during optimization of the network model. I.e., the binary cross entropy cost function:

L(g(z), y)=−y log(g(z))−(1−y)log(1−g(z)) (6)

In Eq. (6), g(z), as an output of the logistic sigmoid activation function from the last layer of the network model, is the prediction of the network model for given input z, and y is the corresponding true label.

However, it should be understood that the specific type of the loss function described above is only an example of this application, and other types of the loss function that conform to the design concept of this application are applicable to this application.

After training the network model is completed, the parameters of the network model and the network structure of the network model are completely saved as an h5 format file. When subsequently used for prediction of new MEG data, it is only necessary to load the format file to predict the multichannel data epoch, and obtain the detection result of whether there is the epileptic spike.

In an embodiment of the present application, the method further includes preprocessing raw sample data to obtain the sample data, the preprocessing includes removing artifacts and normalizing the raw data, and the removing artifacts includes at least one of the followings: removing noises; correcting a baseline drift of the raw data; and removing electrooculogram and electrocardiograph artifacts.

This step of this embodiment is the same as the relevant step described in the embodiments of the method for detecting the epileptic spike. The specific explanation of this step may not be repeated here. Please refer to the relevant description of the relevant step described in the embodiments of the method for detecting the epileptic spike.

Hereinafter, a computer device according to an embodiment of the present application is described with reference to FIG. 13.

FIG. 13 is a block diagram of a computer device according to an embodiment of the present application.

As shown in FIG. 13, the computer device 1300 includes one or more processors 1301 and a memory 1302.

A processor 1301 may be a Central Processing Unit (CPU) or a processing unit which is in other forms and has a data processing capability and/or an instruction execution capability, and may control other components in the computer device 1300 to perform the method of the above embodiments.

The memory 1302 may include one or more computer program products which may include various forms of computer readable storage medium, such as a volatile memory and/or a nonvolatile memory. The volatile memory may include, for example, a Random Access Memory (RAM) and/or a cache, or the like. The nonvolatile memory may include, for example, a Read-Only Memory (ROM), a hard disk, a flash memory, and the like. One or more computer program instructions may be stored in the computer readable storage medium, and the processor 1301 may run the program instructions to implement the method and/or other desired functions of various embodiments of the present application described above. Various contents such as an input signal, a signal component, a noise component and the like may also be stored in the computer readable storage medium.

In an example, the computer device 1300 may further include an input device 1303 and an output device 1304 that are interconnected by a bus system and/or a connection mechanism in other forms (not shown).

The input device 1303 may further include, for example, a keyboard, a mouse and the like.

The output device 1304 may output various information to the outside, including MEG data information, EEG data information, and the like. The output device 1304 may include, for example, a display, a speaker, a printer, a communication network and a remote output device which is connected to the communication network, and the like.

Certainly, for the sake of simplicity, only some of the components of the computer device 1300 related to the present application are shown in FIG. 13, and components such as a bus, an input/output interface, and the like are omitted. In addition, the computer device 1300 may further include any other suitable components according to specific application situations.

In addition to the method and device described above, embodiments of the present application may also be a computer program product including computer program instructions that, when executed by a processor, cause the processor to perform the steps in the method according to various embodiments of the present application described above.

The computer program product may program a program code for performing the operations of the embodiments of the present application in any combination of one or more programming languages. The programming language includes an object-oriented programming language, such as Java, C++, and the like, and further includes a conventional procedural programming language, such as “C” language or similar programming languages. The program code may be executed entirely on a user computing device, be executed partially on a user device, be executed as a separate software package, be executed partially on the user computing device and partially on a remote computing device, or be executed entirely on the remote computing device or a server.

In addition, embodiments of the present application may also be a computer readable storage medium storing computer program instructions that, when executed by a processor, cause the processor to perform the steps in the method according to various embodiments of the present application described above.

The computer readable storage medium may adopt any combination of one or more readable medium. The readable medium may be a readable signal medium or a readable storage medium. The readable storage medium may include, but not limited to, for example, electronic, magnetic, optical, electromagnetic, infrared or a semiconductor system, apparatus or device, or any combinations of the above. More specific examples (non-exhaustive list) of the readable storage medium may include an electrical connection having one or more wires, a portable disk, a hard disk, a Random Access Memory (RAM), a Read-Only Memory (ROM), an Erasable Programmable Read-Only Memory (EPROM or a Flash memory), an optical fiber, a Portable Compact Disc Read-Only Memory (CD-ROM), an optical storage device, a magnetic storage device, or any suitable combination of the above.

The basic principles of the present application have been described above in connection with specific embodiments. However, it needs to be noted that merits, advantages, effects, and the like mentioned in the present application are merely exemplary and not restrictive, and the merits, advantages, effects, and the like are not considered to be requisite in embodiments of the present application. In addition, the specific details of the above application are only for the purpose of illustration and ease of understanding, and are not for a limiting purpose. The above details do not limit the present application must be implemented with the above specific details.

The block diagrams of components, apparatuses, devices and systems mentioned in the present application are merely exemplary examples, and not intended to require or imply that connection, arrangement and configuration must be performed in the manners shown in the block diagrams. As will be appreciated by those skilled in the art, these components, apparatus, devices and systems may be connected, arranged, and configured in any manner. Words such as “including”, “comprising”, “having”, and the like are open-ended words, referring to “including but not limited to”, and may be used therewith interchangeably. The word “or” and “and” used herein refer to a word “and/or”, and may be used therewith interchangeably unless the context indicates otherwise clearly. The word “such as” used herein refers to a phrase “such as but not limited to”, and may be used therewith interchangeably.

It needs to be noted that in the devices, apparatuses and methods of the present application, various components or steps may be divided and/or re-combined. These divisions and/or re-combinations should be considered as equivalent solutions of the present application.

The above description of the disclosed aspects is provided to enable any person skilled in the art to make or use the present application. Various modifications to these aspects are obvious to those skilled in the art, and the general principles defined herein may be applied to other aspects without departing from the scope of the application. Therefore, the present application is not intended to be limited to the aspects shown herein, but covers the broadest scope consistent with the principles and novel features disclosed herein.

The foregoing description has been presented for purposes of illustration and description. In addition, the description is not intended to limit the embodiments of the present application to the forms disclosed herein. Although multiple example aspects and embodiments have been discussed above, those skilled in the art will recognize certain variations, modifications, changes, additions and sub-combinations thereof.

What is claimed is:

1. A method for detecting an epileptic spike, comprising:
obtaining, by a first module of a network model, a local feature of data to be detected, and obtaining, by a second module of the network model, a global feature of the data to be detected; and
determining, by a third module of the network model, a detection result of whether there is the epileptic spike in the data to be detected according to the local feature and the global feature,
wherein the data to be detected contains a temporal domain and a spatial domain represented by multiple channels, the local feature is a single channel feature, and the global feature is a multichannel feature.

2. The method according to claim 1, further comprising:
segmenting the data to be detected to obtain a plurality of multichannel data epochs with a specified time width and a specified number of channels, and each multichannel data epoch of the plurality of multichannel data epochs comprising a plurality of single-channel data epochs with the specified time width,
wherein the obtaining, by a first module of a network model, a local feature of data to be detected comprises:
obtaining, by the first module, a first local feature of each of the plurality of single-channel data epochs; and
sequentially combining, by the first module, the first local feature of each of the plurality of single-channel data epochs according to a position of each channel of the multiple channels to obtain the local feature of the multichannel data epoch,
the obtaining, by a second module of the network model, a global feature of the data to be detected comprises:
obtaining, by the second module, the global feature of the multichannel data epoch, and
the determining, by a third module of the network model, a detection result of whether there is the epileptic spike in the data to be detected according to the local feature and the global feature comprises:
inputting the local feature and the global feature into the third module for weighted combination to obtain a combining feature; and
outputting, by the third module, the detection result of whether there is the epileptic spike in the multichannel data epoch according to the combining feature.

3. The method according to claim 2, wherein the segmenting the data to be detected to obtain a plurality of multichannel data epochs with a specified time width and a specified number of channels comprises:
as for the temporal domain, segmenting, by using the specified time width corresponding to duration of the epileptic spike, the data to be detected into a plurality of data epochs with the specified time width; and
as for the spatial domain, dividing, by using the specified number of channels, the plurality of data epochs according to an anatomical structure of a brain to obtain the plurality of multichannel data epochs.

4. The method according to claim 2, wherein two adjacent multichannel data epochs in the plurality of multichannel data epochs overlap each other at a specified time interval.

5. The method according to claim 2, further comprising:
determining, by a peak detection model, a peak detection result at a peak of the epileptic spike in the multichannel data epoch according to the detection result; and
determining, by a source localization algorithm, a localization of the epileptic spike in a brain according to the peak detection result at the peak of the epileptic spike,
wherein the peak detection model comprises a first peak detection module, a second peak detection module and a third peak detection module, the first peak detection module is used to output the single channel feature of the multichannel data epoch, the second peak detection module is used to output the multichannel feature of the multichannel data epoch, and the third peak detection module is used to output the peak detection result at the peak of the epileptic spike in the multichannel data epoch according to the single channel feature and the multichannel feature.

6. The method according to claim 1, further comprising:
preprocessing raw data to obtain the data to be detected,
wherein the preprocessing comprises removing artifacts and normalizing the raw data.

7. The method according to claim 6, wherein the removing artifacts comprises at least one of the followings:
removing noises;
correcting a baseline drift of the raw data; and
removing electrooculogram and electrocardiograph artifacts.

8. The method according to claim 1, wherein the data to be detected is MEG data or EEG data.

9. A method for training a network model, comprising:
obtaining sample data, the sample data comprising a label with an epileptic spike and a non-epileptic spike; and
training the network model based on the sample data,
wherein the network model comprises a first module configured to output a local feature of the sample data, a second module configured to output a global feature of the sample data, and a third module configured to output a training result of whether there is the epileptic spike in the sample data according to the local feature and the global feature, and the sample data to be detected contains a temporal domain and a spatial domain represented by multiple channels, the local feature is a single channel feature, and the global feature is a multichannel feature.

10. The method according to claim 9, further comprising:
segmenting the sample data to obtain a plurality of multichannel sample data epochs with a specified time width and a specified number of channels, each multichannel sample data epoch of the plurality of multichannel sample data epochs comprising a plurality of single-channel sample data epochs with the specified time width,
wherein the training the network model based on the sample data comprises:
inputting the multichannel sample data epoch into the first module to obtain the local feature, and inputting the multichannel sample data epoch into the second module to obtain the global feature;
outputting, by the third module, the training result of whether there is the epileptic spike in the multichannel sample data epoch according to the local feature and the global feature; and
updating parameters of the network model according to a difference between the training result and the label.

11. The method according to claim 10, wherein the first module comprises a one-dimensional convolutional neural network, and
the inputting the multichannel sample data epoch into the first module to obtain the local feature comprises:
obtaining, by the one-dimensional convolutional neural network, a first local feature of each of the plurality of single-channel sample data epochs; and
sequentially combining the first local feature of each of the plurality of single-channel sample data epochs according to a position of each channel of the multiple channels to obtain the local feature of the multichannel sample data epoch.

12. The method according to claim 10, wherein the second module comprises an unshared weight two-dimensional convolutional neural network and a shared weight two-dimensional convolutional neural network, and
the inputting the multichannel sample data epoch into the second module to obtain the global feature comprises:
inputting the multichannel sample data epoch into the unshared weight two-dimensional convolutional neural network to obtain a first global feature of the multichannel sample data epoch;
inputting the first global feature of the multichannel sample data epoch into the shared weight two-dimensional convolutional neural network to obtain a second global feature of the multichannel sample data epoch; and
obtaining the global feature according to the second global feature.

13. The method according to claim 10, wherein the outputting, by the third module, the training result of whether there is the epileptic spike in the multichannel sample data epoch according to the local feature and the global feature comprises:
performing weighted combination on the local feature and the global feature to obtain a combining feature; and
outputting the training result of whether there is the epileptic spike in the multichannel sample data epoch according to the combining feature.

14. The method according to claim 10, wherein the segmenting the sample data to obtain a plurality of multichannel sample data epochs comprises:
as for the temporal domain, segmenting, by using the specified time width corresponding to duration of the epileptic spike, the sample data into a plurality of sample data epochs with the specified time width; and
as for the spatial domain, dividing, by using the specified number of channels, the plurality of sample data epochs according to an anatomical structure of a brain to obtain the plurality of multichannel sample data epochs.

15. The method according to claim 10, wherein two adjacent multichannel sample data epochs in the plurality of multichannel sample data epochs overlap each other at a specified time interval.

16. The method according to claim 10, wherein a loss function of the network model is a binary cross entropy loss function, and
the updating parameters of the network model according to a difference between the training result and the label comprises:
obtaining a loss function value according to the binary cross entropy loss function and the difference between the training result and the label; and
updating the parameters of the first module, the second module and the third module according to the loss function value.

17. The method according to claim 9, further comprising:
preprocessing raw sample data to obtain the sample data,
wherein the preprocessing comprises removing artifacts and normalizing the raw data.

18. The method according to claim 17, wherein the removing artifacts comprises at least one of the followings:
removing noises;
correcting a baseline drift of the raw data; and
removing electrooculogram and electrocardiograph artifacts.

19. A computer device, comprising:
a memory, a processor and a computer program stored on the memory and executed by the processor,
wherein when the processor executes the computer program, the processor performs the following steps:
obtaining, by a first module of a network model, a local feature of data to be detected, and obtaining, by a second module of the network model, a global feature of the data to be detected; and
determining, by a third module of the network model, a detection result of whether there is the epileptic spike in the data to be detected according to the local feature and the global feature,
wherein the data to be detected contains a temporal domain and a spatial domain represented by multiple channels, the local feature is a single channel feature, and the global feature is a multichannel feature.

20. The computer device according to claim 19, wherein the processor further performs the following steps:
obtaining sample data, the sample data comprising a label with an epileptic spike and a non-epileptic spike; and
training the network model based on the sample data,
wherein the network model comprises a first module configured to output a local feature of the sample data, a second module configured to output a global feature of the sample data, and a third module configured to output a training result of whether there is the epileptic spike in the sample data according to the local feature and the global feature, and the sample data to be detected contains a temporal domain and a spatial domain represented by multiple channels, the local feature is a single channel feature, and the global feature is a multichannel feature.
